(19) United States
(12) Patent Application Publication
(10) Pub. No.: US 2024/0386564 A1
(43) Pub. Date: Nov. 21, 2024

(54) IMAGE PROCESSING APPARATUS, IMAGE PROCESSING METHOD, AND MEDIUM

(71) Applicant: CANON KABUSHIKI KAISHA, Tokyo (JP)

(72) Inventor: Toshimizu YAMANE, Tokyo (JP)

(21) Appl. No.: 18/659,142

(22) Filed: May 9, 2024

(30) Foreign Application Priority Data
May 17, 2023 (JP) 2023-081469

(51) Int. Cl. G06T 7/00; G16H 30/40

(52) U.S. Cl. CPC G06T 7/0016; G16H 30/40; G06T 2207/20224; G06T 2207/30096

(57) ABSTRACT

To efficiently select an image to be used for generation of a subtraction image, provided is an image processing apparatus including: a first image acquiring unit configured to acquire first image data which is image data obtained by imaging a subject by a first examination; and a comparison image acquiring unit configured to acquire comparison image data for performing comparison to the first image data, the comparison image acquiring unit being configured to acquire the comparison image data in accordance with priority with respect to a combination including the first image data and a candidate for the comparison image data, the priority being calculated based on an examination date/time of the first examination and a contrast enhancement state of the first image data.

### BACKGROUND OF THE INVENTION

### Field of the Invention

The present disclosure relates to an image processing apparatus, an image processing method, and a medium.

### Description of the Related Art

In the medical field, diagnosis is performed through use of images acquired by various medical image acquiring apparatus (modalities) such as a computed tomography photographing apparatus (hereinafter referred to as “computed tomography (CT) apparatus”). In order to efficiently perform diagnosis, a technology of supporting doctor's diagnosis by visualizing temporal change of a lesion or a contrast effect obtained by a contrast agent is required.

As a technology of visualizing the temporal change, a temporal subtraction image calculating technology has been known. In this technology, two images (for example, a present image and a past image) of the same patient taken at different times are subjected to registration so that a subtraction image that visualizes a difference between those images is calculated. Comparison between images can be supported by displaying the temporal subtraction image calculated in this manner. An examination that becomes a reference of the calculation of the difference between the images is referred to as “target examination.” An image taken in the target examination corresponds to the above-mentioned present image, and an image of the same patient as a patient being a target of the target examination, which has been taken in a past examination performed before the target examination, corresponds to the past image.

Meanwhile, as a technology of visualizing the contrast effect obtained by the contrast agent, a contrast phase subtraction image calculating technology (Atsushi Urikura, “Utilizing characteristics of contrast CT images for accurate image diagnosis” Advanced Imaging Seminar 2019 (May 2019 issue)), Internet <URL: https://www.innervision.co.jp/sp/ad/suite/canonmedical/sup201905/session2-1 ct3> has been known. In this technology, a difference between a contrast image and a non-contrast image is visualized so that a portion of a tumor or invasion can be easily seen. This technology utilizes the characteristics in which a cancer cell performs cell division more actively as compared to other cells and thus requires a larger amount of oxygen and nutrition. With the characteristics, the tumor or the invasion has a larger amount of blood flow, and a larger amount of contrast agent is introduced therein. Thus, the contrast effect appears stronger than other sites. The contrast phase subtraction image calculating technology includes a method of subtracting the non-contrast image from the contrast image, and a method of performing subtraction or the like between contrast images having different phases such as an early phase and a late phase.

In order to visualize occurrence of a lesion such as a tumor or invasion through use of the above-mentioned temporal subtraction image calculating technology, both of the present image and the past image are required. Meanwhile, in order to visualize the lesion through use of the contrast phase subtraction image calculating technology, both of the contrast image and the non-contrast image are required. Hitherto, a user has been required to check presence or absence of the past image or the contrast image and to appropriately select, from among a plurality of medical images, an image conforming to an examination object and being used for generation of the subtraction image. Further, at this time, the user has been required to select whether to perform temporal subtraction image calculating processing or contrast phase subtraction image calculating processing each time in consideration of the image or the like. Accordingly, selection of an image to be used for generation of the subtraction image has been work that requires a troublesome effort for the user.

### SUMMARY OF THE INVENTION

The present disclosure has been made in view of the above-mentioned circumstances, and has an object to provide an image processing apparatus with which an image to be used for generation of a subtraction image can be efficiently selected.

In order to achieve the above-mentioned object, according to one aspect of the present disclosure, there is provided an image processing apparatus including: a first image acquiring unit configured to acquire first image data which is image data obtained by imaging a subject by a first examination; and a comparison image acquiring unit configured to acquire comparison image data for performing comparison to the first image data, the comparison image acquiring unit being configured to acquire the comparison image data in accordance with priority with respect to a combination including the first image data and a candidate for the comparison image data, the priority being calculated based on an examination date/time of the first examination and a contrast enhancement state of the first image data.

Further features of the present invention will become apparent from the following description of exemplary embodiments with reference to the attached drawings.

### BRIEF DESCRIPTION OF THE DRAWINGS

FIG. 1 is a diagram for schematically illustrating an overall configuration of an image processing apparatus according to a first embodiment.

FIG. 2 is a flow chart for illustrating all steps to be executed in the image processing apparatus illustrated in FIG. 1.

FIG. 3 is a flow chart for illustrating each step in processing of generating an image pair.

FIG. 4A is an explanatory view for illustrating a method of determining an image pair, and is a view for schematically illustrating a case in which a contrast image and a non-contrast image are mainly shown with a date/time being changed.

FIG. 4B is an explanatory table for illustrating the method of determining an image pair, and is a table for showing a relationship between the image pair and priority.

FIG. 4C is an explanatory table for illustrating the method of determining an image pair, and is a table for showing the relationship between the image pair and the priority.

FIG. 5 is a flow chart for illustrating each step in processing of generating an image pair to be executed in image processing in Modification Example 2 of the first embodiment.

FIG. 6 is a flow chart for illustrating each step in processing of generating a subtraction image to be executed in an image processing apparatus according to a second embodiment.

FIG. 7A is a flow chart for illustrating each step in the processing of generating a subtraction image.

FIG. 7B is a flow chart for illustrating each step in the processing of generating a subtraction image.

FIG. 8 is a diagram for schematically illustrating an overall configuration of an image processing apparatus according to a third embodiment.

FIG. 9 is a flow chart for illustrating all steps to be executed in the image processing apparatus illustrated in FIG. 8.

FIG. 10A is a flow chart for illustrating each step in processing of generating a subtraction image with evaluation value.

FIG. 10B is a flow chart for illustrating each step in processing of generating a subtraction image with evaluation value.

FIG. 11 is a diagram for schematically illustrating an overall configuration of an image processing apparatus according to a fourth embodiment.

FIG. 12 is a flow chart for illustrating all steps to be executed in the image processing apparatus illustrated in FIG. 11.

FIG. 13 is a flow chart for illustrating each step in processing of displaying a confirmation dialog.

FIG. 14 is a flow chart for illustrating each step in callback processing at the time of displaying an image list.

FIG. 15 is a flow chart for illustrating each step in image display processing.

FIG. 16 is a view for illustrating an example of the confirmation dialog.

FIG. 17 is a view for illustrating an example of the image list.

FIG. 18 is a view for illustrating an example of an image detail screen.

### DESCRIPTION OF THE EMBODIMENTS

The present disclosure is described in detail based on exemplary embodiments thereof with reference to the attached drawings. The configurations of the embodiments described below are only examples, and the present disclosure is not limited to the illustrated configurations. A plurality of features are described in the embodiments, but the present disclosure does not necessarily require all of those plurality of features, and a plurality of features may be combined as appropriate. Further, in the attached drawings, the same or similar components are denoted by the same reference symbols, and redundant description thereof is omitted.

### First Embodiment

An image processing apparatus according to a first embodiment compares a plurality of images of a target patient to acquire a subtraction image for visualizing a lesion of the target patient. The image processing apparatus selectively acquires an image to be used for generation of the subtraction image appropriate for visualizing the lesion, from a temporal subtraction image or a contrast phase subtraction image of the target patient. In the present disclosure, the temporal subtraction image is a subtraction image between a present image and a past image. Further, the contrast phase subtraction image is a subtraction image between a contrast phase image and a non-contrast phase image, or a subtraction image between contrast images having different phases such as an early phase and a later phase. The present disclosure relates to a method of selecting an image to be used for generation of the subtraction image, but generation of the subtraction image is also included in one mode of the present disclosure.

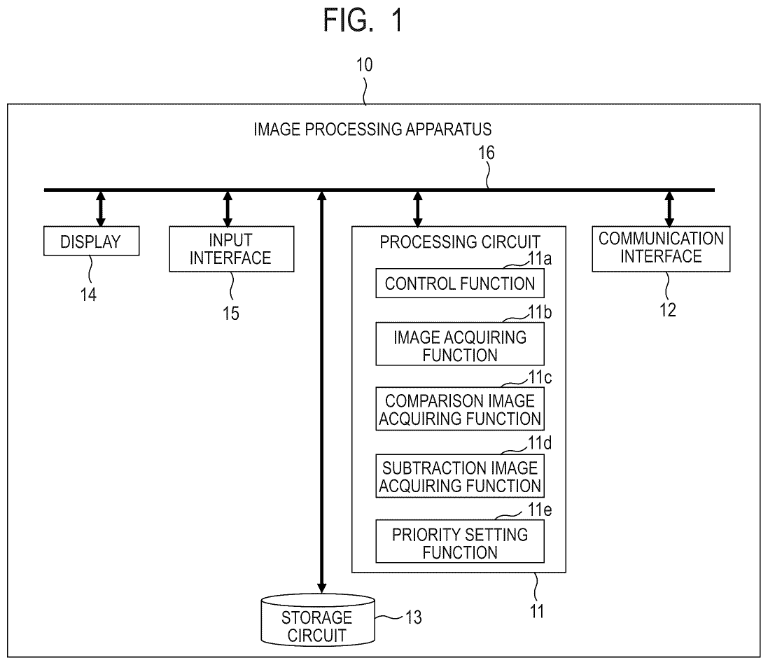

FIG. 1 is a diagram for illustrating an example of a configuration of an image processing apparatus 10 according to the first embodiment. As illustrated in FIG. 1, the image processing apparatus 10 includes a processing circuit 11, a communication interface 12, a storage circuit 13, a display 14, an input interface 15, and a connection portion 16. Further, the image processing apparatus 10 is connected to a medical image diagnosis apparatus (modality), a medical image storage apparatus, each department system, and the like via a network (not shown) so as to allow communication therebetween.

The medical image diagnosis apparatus includes an X-ray computed tomography (CT) apparatus, a magnetic resonance imaging (MRI) apparatus, and an X-ray diagnosis apparatus. The medical image storage apparatus is implemented by a picture archiving and communication system (PACS) or the like. In addition, the medical image storage apparatus stores a medical image taken by the medical image diagnosis apparatus in a format complying with digital imaging and communication medicine (DICOM). The department systems include, for example, a hospital information system (HIS) and a radiology information system (RIS). The department systems further include various systems such as a diagnosis report system and a laboratory information system (LIS).

The processing circuit 11 in the first embodiment includes a control function 11a, an image acquiring function 11b, a comparison image acquiring function 11c, a subtraction image acquiring function 11d, and a priority setting function 11e. In addition, the processing circuit 11 controls the image processing apparatus 10 by executing those functions in accordance with an input operation received from a user via the input interface 15. In this case, the image acquiring function 11b is an example of an image acquiring unit. Further, the comparison image acquiring function 11c is an example of a comparison image acquiring unit. Further, the subtraction image acquiring function 11d is an example of a subtraction image acquiring unit. Further, the priority setting function 11e is an example of a priority setting unit.

In accordance with operations input via the input interface 15, the control function 11a executes control of generating various graphical user interfaces (GUIs) or various kinds of display information and displaying the GUIs and the information on the display 14. Further, the control function 11a controls transmission/reception of information to/from an apparatus or a system on a network (not shown) via the communication interface 12. Specifically, the control function 11a controls acquisition of three-dimensional medical images (volume data) from the medical image diagnosis apparatus, the medical image storage apparatus, or the like connected to the network. Further, the control function 11a controls acquisition of information regarding a subject from an external system connected to the network. Further, the control function 11a controls output of processing results to the apparatus or system on the network.

The image acquiring function 11b acquires image data obtained by imaging the subject by the medical image diagnosis apparatus or the like from the medical image storage apparatus or the like. At this time, the image data is acquired based on reference address information (uniform resource locator (URL), various unique identifiers (UIDs), path string, or the like) of the image. Processing to be performed by the image acquiring function 11b is described in detail later.

The comparison image acquiring function 11c selects, from an image list of a target patient to be described later, an image (comparison image) for use in comparison to an image of a target examination. Processing to be performed by the comparison image acquiring function 11c is described in detail later.

The subtraction image acquiring function 11d acquires a subtraction image between the image of the target examination and the comparison image. Processing to be performed by the subtraction image acquiring function 11d is described in detail later.

The priority setting function 11e refers to an examination date/time or an examination object to set priority of, as a combination of the image of the target examination and an image that is planned to be acquired as a comparison target image, a pair of those two images. Processing to be performed by the priority setting function 11e is described in detail later. The examination date/time also includes information (examination date) of only the date of an examination, which does not include time information.

The above-mentioned processing circuit 11 is implemented by, for example, a processor. In this case, each of the above-mentioned processing functions is stored in the storage circuit 13 in a mode of a program executable by a computer. Further, the processing circuit 11 implements a function corresponding to each program by reading out each program stored in the storage circuit 13 and executing the program. In other words, the processing circuit 11 includes each processing function illustrated in FIG. 1 under a state in which each program is read out.

The processing circuit 11 may be formed by combining a plurality of independent processors, and each processor may implement each processing function by executing the program. Further, the processing functions included in the processing circuit 11 may be implemented by appropriately distributing or integrating those processing functions to a single or a plurality of processing circuits. Further, the processing functions included in the processing circuit 11 may be implemented by a combination of software and hardware such as a circuit. Further, description has been given here of an example of a case in which the programs corresponding to the respective processing functions are stored in the single storage circuit 13, but the embodiment is not limited thereto. For example, the programs corresponding to the respective processing functions may be distributed and stored in a plurality of storage circuits, and the processing circuit 11 may read out each program from each storage circuit and execute the program.

The communication interface 12 controls transmission and communication of various kinds of data transmitted or received between the image processing apparatus 10 and another apparatus or system connected via the network. Specifically, the communication interface 12 is connected to the processing circuit 11, and outputs data received from another apparatus or system to the processing circuit 11, or transmits data output from the processing circuit 11 to another apparatus or system. For example, the communication interface 12 is implemented by a network card, a network adapter, a network interface controller (NIC), or the like.

The storage circuit 13 stores various kinds of data and various programs. Specifically, the storage circuit 13 is connected to the processing circuit 11, and stores data input from the processing circuit 11 or reads out stored data to output the data to the processing circuit 11. For example, the storage circuit 13 is implemented by a random access memory (RAM), a semiconductor memory element such as a flash memory, a hard disk, an optical disc, or the like.

The display 14 displays various kinds of information and various kinds of data. Specifically, the display 14 is connected to the processing circuit 11, and displays various kinds of information and various kinds of data output from the processing circuit 11. For example, the display 14 is implemented by a liquid crystal display, a cathode ray tube (CRT) display, an organic EL display, a plasma display, a touch panel, or the like.

The input interface 15 receives operations of inputting various instructions and various kinds of information from the user. Specifically, the input interface 15 is connected to the processing circuit 11, and converts an input operation received from the user into an electrical signal and outputs the electrical signal to the processing circuit 11. For example, the input interface 15 is implemented by a trackball, a switch button, a mouse, a keyboard, a touchpad on which an input operation is performed by touching an operation surface, or a touch screen formed by integrating a display screen and a touchpad. Further, the input interface 15 is implemented also by a non-contact input interface using an optical sensor, a voice input interface, or the like. In this specification, the input interface 15 is not limited to only an interface including a physical operation component such as a mouse or a keyboard. For example, an electrical signal processing circuit for receiving an electrical signal corresponding to an input operation from an external input device provided separately from the apparatus and outputting the electrical signal to a control circuit is also included as an example of the input interface 15.

The connection portion 16 is a bus or the like for connecting the processing circuit 11, the communication interface 12, the storage circuit 13, the display 14, and the input interface 15 to each other.

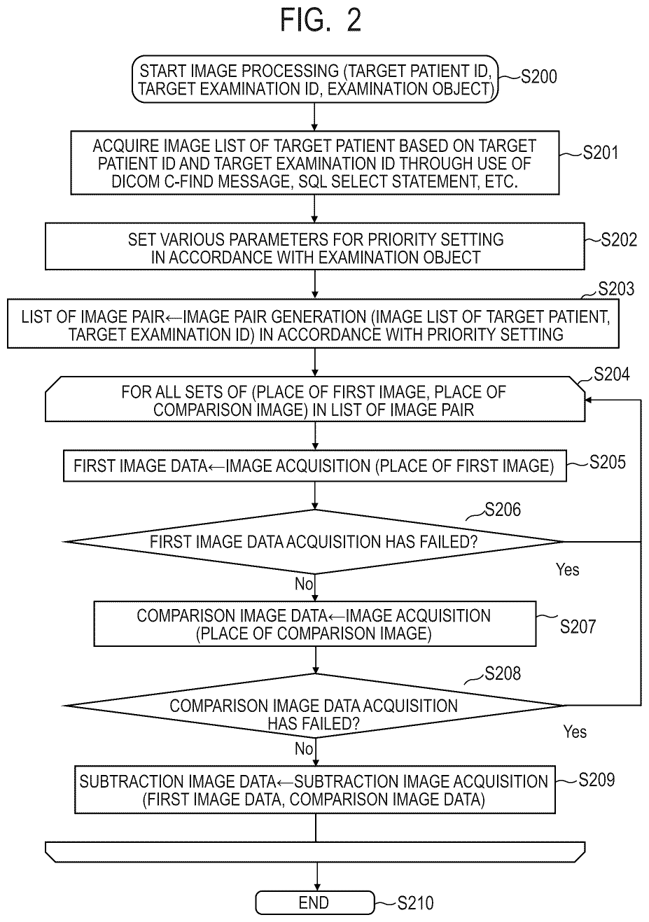

FIG. 2 is a flow chart for illustrating all steps of processing to be executed in the image processing apparatus 10 according to the first embodiment. The image processing apparatus 10 inputs a target patient ID for identifying an image processing target, a target examination ID, and an examination object. Then, the image processing apparatus 10 acquires, based on those inputs, first image data selected from pieces of image data of the target examination and comparison image data which is image data for use in comparison to the first image data, and outputs subtraction image data for visualizing a lesion.

In this case, all pieces of image data are three-dimensional volume data. In the first embodiment, description is given assuming that the target examination is an X-ray CT examination, and the first image data and the comparison image data are both X-ray CT images. However, the embodiment of the present disclosure is not limited thereto, and the present disclosure is also applicable to image data of other modalities, such as magnetic resonance imaging (MRI) images or ultrasonic images. Further, the first image data and the comparison image data in the first embodiment may be images taken in different examinations, such as a present image and a past image of the target patient, or may be a combination of images other than the above. For example, the first image data and the comparison image data may be two images (for example, contrast image and non-contrast image) taken in the same examination under different contrast conditions. Moreover, for example, the non-contrast image may be a virtual non-contrast CT image generated through use of dual-energy CT or the like.

Next, with reference to the flow chart of FIG. 2, a process of generating a subtraction image to be executed in the first embodiment is described. For example, as illustrated in FIG. 2, in the first embodiment, image processing is started in response to, as inputs, a target patient ID of a certain subject, a target examination ID, and an examination object, which are determined through an instruction operation or the like input via the input interface 15 (Step S200). In the first embodiment, as the “examination object,” the user makes selection via the input interface 15 from prescribed options such as “visualization of invasion in spinal canal,” “visualization of liver tumor,” and the like so that information indicating which of those options is the object of this examination is acquired. The examination objects given here are examples given in order to facilitate the description below, and publicly-known examination objects using the subtraction image, such as “visualization of bone metastasis,” “visualization of lung metastasis,” and “visualization of brain tumor,” are also included in the options.

The control function 11a retrieves an image (image of the target examination) taken in an examination (target examination) indicated by the target examination ID and an image of a past examination relating to the patient (target patient) indicated by the target patient ID. Specifically, the control function 11a retrieves, through use of C-FIND message of the DICOM protocol or SELECT statement of SQL, image data which is associated with the target patient ID and has an examination date that is the same as the examination date of the target examination ID or has a past examination date before the examination date of the target examination ID, and acquires an image list of the target patient (Step S201).

Next, the priority setting function 11e sets various parameters for setting the priority, based on the examination object acquired in Step S200 (Step S202). As specific examples, the parameters include “date/time difference boundary value of examination date/time” used when it is determined whether or not the examination dates/times of the images are sufficiently separated from each other, “date/time large weight” and “date/time small weight” which are weight values weighted on the date/time difference in examination date/time at the time of calculating the priority, and “contrast weight” which is a weight value weighted on a difference in contrast enhancement state (contrast difference).

In Step S202, the priority setting function 11e sets those parameters in accordance with the examination object.

In this case, “date/time large weight” is a weight for raising the priority as the date/time difference is increased when priority is given to generation of the temporal subtraction image, and thus a relatively large value of about 100 is set therefor. Further, “date/time small weight” is a weight for lowering the priority as the date/time difference is increased when priority is given to generation of the contrast phase subtraction image, and thus a relatively small value of about −0.1 is set therefor.

For example, when the examination object is “visualization of invasion in spinal canal,” it is desired to give priority to generation of the temporal subtraction image because the presence or absence of the invasion in the spinal canal clearly appears as a temporal change. Accordingly, “date/time difference boundary value of examination date/time” is set to about one week as a predetermined value, and the weight value of “contrast weight” is set to be smaller than the weight value of “date/time large weight.” In this manner, the priority of an image pair having a large date/time difference in examination date/time and a small difference in contrast enhancement state can be raised.

Further, for example, when the examination object is “visualization of liver tumor,” it is desired to give priority to generation of the contrast phase subtraction image because the presence or absence of the liver tumor is clearly rendered as a difference of a contrast effect. Accordingly, “date/time difference boundary value of examination date/time” is set to about two days as a predetermined value, and the weight value of “contrast weight” is set to be larger than the weight value of “date/time large weight.” In this manner, the priority of an image pair that is taken on the same date and has a difference in contrast enhancement state can be raised.

Further, no matter which of the above-mentioned examination objects is selected, the weight value of “date/time large weight” is set to a value sufficiently larger than the weight value of “date/time small weight.” Further, it is desired that “date/time difference boundary value of examination date/time” be set to a small value as a weight value when it is predicted that the progression rate of a tumor or invasion is fast, such as when the age of the patient is young.

In this case, the user sets the parameters (weight values) for determining the priority in accordance with the examination object, but the method of setting the parameters is not limited to a method performed by the user.

For example, numerical values for weighting (weight values) determined in advance may be stored in advance in the storage circuit 13 as a table in accordance with the examination object, the date/time difference, the contrast difference, and the like, and those numerical values may be read out as appropriate in accordance with the image pair to be used for priority calculation to be described later. The contrast difference as described here is obtained by quantifying a difference in phase of contrast imaging. Further, one embodiment of the present invention may also encompass a case in which the priority setting function 11e automatically sets “date/time difference boundary value of examination date/time” in accordance with the age of the patient. When the patient is young, in general, it is considered that tumor growth or the like is fast. Thus, it is desired that “date/time difference boundary value of examination date/time” be set to be shorter as the age of the patient becomes younger.

Next, the comparison image acquiring function 11c acquires the list of the image pair which is a combination including a first image and a comparison image, in accordance with the priority based on at least one of the examination date/time or the contrast enhancement state (Step S203). That is, the comparison image acquiring function 11c selects the first image from images of the target examination in the image list of the target patient, and selects the comparison image from images other than the first image in the image list of the target patient.

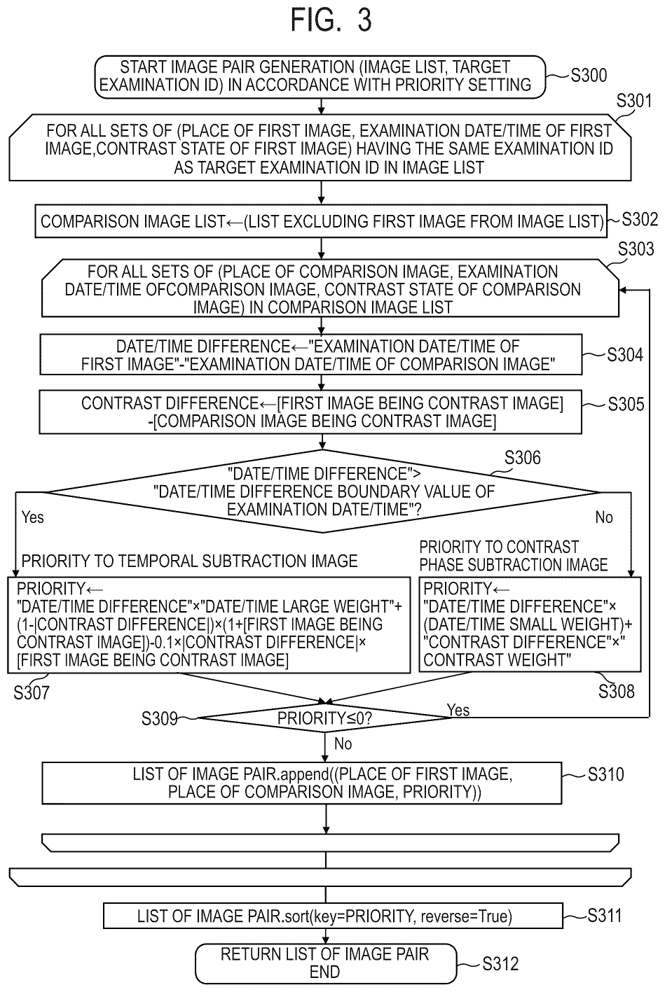

Now, sub-steps of image pair generation to be executed in Step S203 are described with reference to the flow chart of FIG. 3 from Step S300 to Step S312. In Step S301, specifically, each of all images having the same examination ID as the target examination ID in the image list obtained in Step S201 (that is, images of the target examination) is a candidate for the first image. In addition, through use of information (place of image, examination date/time, contrast enhancement state) of this candidate for the first image as information of a target image, loop processing of from Step S302 to Step S310 is executed by the comparison image acquiring function 11c.

In this case, the comparison image acquiring function 11c selects the first image from among all of the images of the target examination in the image list of the target patient. However, the first embodiment is not limited thereto. For example, the user may select one or a plurality of images suitable for the subtraction image to be obtained from among the images of the target examination displayed on the display 14 in advance. As another example, for example, a selection condition conforming to the object, such as an imaging site or a reconstruction function, may be determined in advance, and the comparison image acquiring function 11c may select one or a plurality of images from the images of the target examination in accordance with this selection condition. In the actual loop processing, first, a list excluding the first image from the image list is acquired as a comparison image list (Step S302). Next, for each of all images in the comparison image list as a candidate for the comparison image, information (for example, place of the image, examination date/time, contrast enhancement state) of this candidate for the comparison image is subjected to loop processing of from Step S304 to Step S310 (Step S303). First, a date/time difference is calculated by subtracting the examination date/time of the comparison image from the examination date/time of the first image (Step S304). In this case, the date/time difference may be a difference in examination date or a difference in examination time.

Next, a contrast difference is calculated by subtracting [comparison image being contrast image] from [first image being contrast image] (Step S305). In this case, [proposition] is in Iverson bracket, which is 1 when the proposition is true and 0 when the proposition is false. As the method of calculating the contrast difference, other methods may be used as long as the difference in phase is quantified, such as a difference in elapsed time from contrast agent administration. Next, in Step S306, when the date/time difference is larger than “date/time difference boundary value of examination date/time,” it is determined that priority is given to calculation of the temporal subtraction image as the subtraction image, and the process proceeds to Step S307. Otherwise, it is determined that priority is given to calculation of the contrast phase subtraction image as the subtraction image, and the process proceeds to Step S308.

In Step S307, processing of calculating the priority of the image pair is executed so that priority is given to an image pair having no contrast difference and a larger date/time difference. Specifically, the following calculation is performed.

Priority←“Date/time difference”×“Date/time large weight”+

(1−|Contrast difference|)×(1+[First image being contrast image])−

0.1×|Contrast difference|×[First image being contrast image]

With this calculation, the first term allows an image pair having a larger date/time difference to have a higher priority. Further, when the date/time difference is the same, the second term allows an image pair having no contrast difference to have a higher priority, and further allows, among them, a pair of contrast images to have a higher priority. In a case of an image pair having a contrast difference although the date/time difference is the same, the third term allows a pair having the first image being a contrast image to have a lower priority.

In this manner, there is provided an effect of lowering the priority of the image pair having a low specificity at the time of finding a lesion from a difference between images. The above-mentioned priority calculation expression is an example of a priority calculation expression for giving priority to temporal subtraction image calculation, and other calculation expressions may be used as long as the priority of an image pair having a high specificity can be raised and the priority of an image pair having a low specificity can be lowered.

In Step S308, processing of calculating the priority of the image pair is executed so that priority is given to an image pair having a contrast difference and a smaller date/time difference. Specifically, the following calculation is performed.

Priority←“Date/time difference”×“(Date/time small weight)”+“Contrast difference”×“Contrast weight”

In this manner, the priority of an image pair having a contrast difference can be made higher than the priority of an image pair having no contrast difference. Further, when the contrast difference is the same, the “date/time small weight” is a negative value, and hence the priority of an image pair having a date/time difference can be lowered. Moreover, the priority of an image pair having a small date/time difference and no contrast difference or an image pair of a combination of subtracting the contrast image from the non-contrast image becomes 0 or less. The above-mentioned priority calculation expression is an example of a priority calculation expression of giving priority to contrast phase subtraction image calculation, and other calculation expressions may be used as long as the priority of an image pair having a contrast difference can be raised and the priority of an image pair having no contrast difference can be lowered.

Next, in Step S309, determination is made based on the priority calculated in Step S307 or Step S308. Specifically, when the calculated priority is 0 or smaller, the image combination is determined to be unsuitable for calculation of the subtraction image, and the process returns to Step S303 to proceed to calculation of the priority for the next image pair. When the calculated priority is larger than 0, the process proceeds to Step S310.

In Step S310, information of a combination of the place of the first image, the place of the comparison image, and the priority which have been acquired by the above-mentioned processing steps is appended to the list of the image pair (Step S310). After appending to the list, the flow returns to Step S303, and the priority is obtained for each image pair based on the comparison image list obtained in Step S302 so that the image pair is appended to the list as appropriate.

After the calculation of the priority of all image pairs based on the comparison image list obtained in Step S302 is ended, the flow returns to Step S301, and processing of from Step S302 to Step S310 is performed for a new first image. After the processing of obtaining the priority of the image pair is ended for all images having the same examination ID as the target examination ID and thus the process exits from a double loop, the list of the image pair is sorted in descending order through use of the priority as a key (Step S311).

Those processing steps are achieved by, for example, the processing circuit 11 invoking programs corresponding to the control function 11a, the priority setting function 11e, and the comparison image acquiring function 11c from the storage circuit 13 and executing the programs. With the processing of from Step S300 to Step S312 described above, a list of image pairs arranged in descending order of priority is acquired.

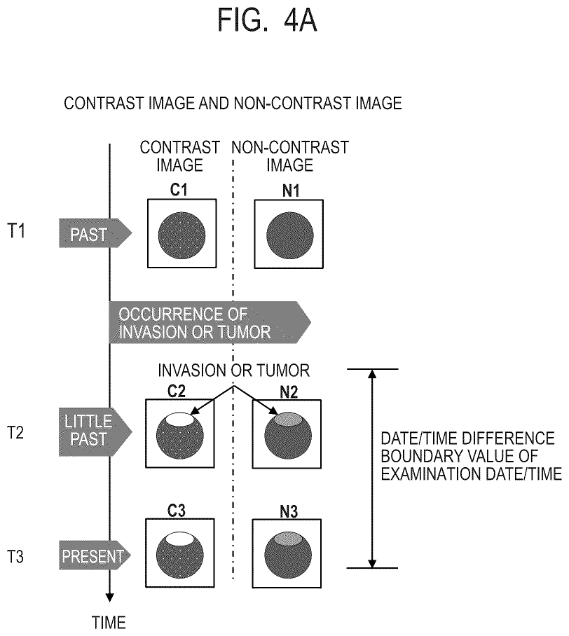

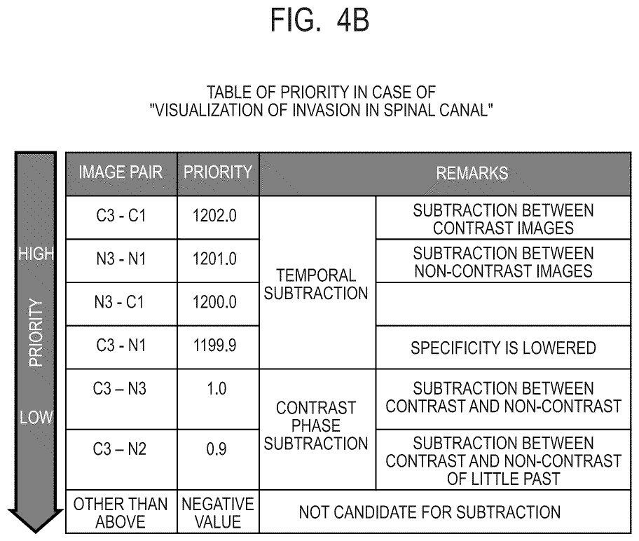

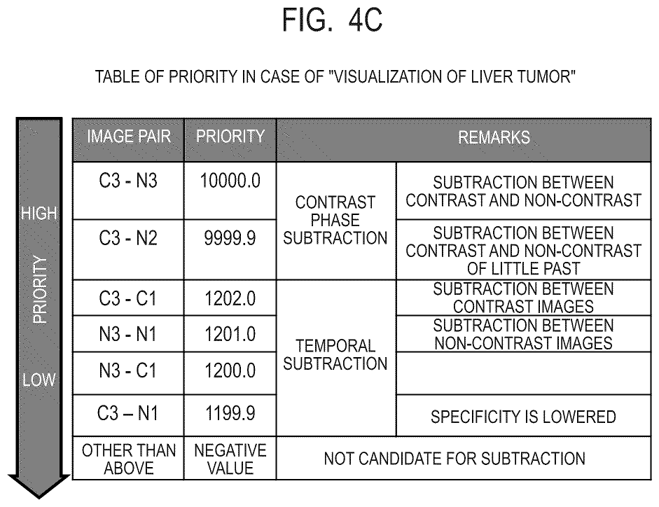

Details of the processing steps of the sub-steps illustrated in FIG. 3, which are executed in Step S203 described above, are described in detail with reference to specific examples illustrated in FIG. 4A to FIG. 4C. Now, description is given of a case in which contrast images and non-contrast images taken for a certain subject are acquired as illustrated in FIG. 4A, and the examination object is “visualization of invasion in spinal canal.”

In FIG. 4A, an examination is performed for a subject having the same examination ID at each of three time points (three dates/times) of T1 (past), T2 (little past), and T3 (present), and a contrast image and a non-contrast image are taken in each examination. Further, a change such as occurrence of invasion or a tumor has occurred in the subject between T1 and T2, and this change appears as a difference between the examination images of T1 and T2. Moreover, an examination is carried out also at a time point of T3 a little after T2, and this date/time difference falls within a range of “date/time difference boundary value of examination date/time.”

First, it is assumed that a contrast image C3 and a non-contrast image N3 are acquired at an examination date/time T3 (=13th day) being a subtraction target. Further, it is assumed that, at a time point of T2 (=12th day) being a time point one day before T3, a contrast image C2 and a non-contrast image N2 are acquired. It is assumed that the examination dates/times of the images C2, C3, N2, and N3 fall within the range of the “date/time difference boundary value of examination date/time” determined in advance from the examination date/time T3 being an examination target. Further, it is assumed that, as an image taken before the time point of occurrence of invasion or a tumor, a contrast image C1 and a non-contrast image N1 are acquired at the examination date/time T1 (=1st day).

As described above as well, the presence or absence of invasion in the spinal canal clearly appears as a temporal change, and hence it is desired to give priority to generation of the temporal subtraction image. In the case of this example, the examination object is “visualization of invasion in spinal canal,” and hence a small contrast weight is set, such as “contrast weight”=1.0. As a result, the list of the image pair with respect to the target examination at the examination date/time T3 becomes, for example, as shown in the table of FIG. 4B, a list in which priority is given to an image pair providing a temporal subtraction, and among such pairs, an image pair (C3-C1) taking a subtraction between contrast images has the highest priority.

In this example, the priority of the contrast phase subtraction (C3-N3, C3-N2) is set to be low. However, for example, when the examination object is “visualization of liver tumor,” the presence or absence of the liver tumor is clearly rendered as a difference of a contrast effect. Thus, in such a case, it is desired to set a large contrast weight, such as “contrast weight”=10,000.0. Results obtained by performing such setting of parameters are shown in the table of FIG. 4C. With such setting, as shown in FIG. 4C, the priority of the contrast phase subtraction can also be raised.

The description of the first embodiment has presented an example of determining the priority in accordance with the presence or absence of the contrast agent or the phase difference, but, in addition to the above, the present invention may determine the priority in accordance with the type of the contrast agent. For example, in a case of MRI image diagnosis of a liver cancer, the priority of an image pair including an examination image using a Primovist contrast agent may be raised. Specifically, as for the contrast weight used at the time of priority calculation performed in Step S307 or Step S308 of FIG. 3, when the Primovist contrast agent is used, multiplication by 1.1 may be performed to raise the priority.

The list of the image pair is obtained by executing the above-mentioned sub-steps in Step S203. The control function 11a repeatedly executes, as illustrated in FIG. 2, the subsequent Step S205 to Step S209 as loop processing for all pieces of pair information (place of first image, place of comparison image) in the obtained list of the image pair, in order from the pair information having a higher priority (Step S204). Those processing steps are achieved by, for example, the processing circuit 11 invoking programs corresponding to the control function 11a and the image acquiring function 11b from the storage circuit 13 and executing the programs.

In the loop processing, the image acquiring function 11b acquires the image data from the place of the first image (Step S205). Specifically, the image acquiring function 11b acquires the image data through use of C-MOVE/C-STORE message of the DICOM protocol, in accordance with the place of the image. As another example, a hypertext transfer protocol (HTTP), a server message block (SMB), file input/output, or the like can be used to acquire the image data. In this case, when the acquisition of the first image data has failed due to a communication error, a file I/O error, or the like, the process returns to Step S204, and the loop processing is continued for the next pair in the list of the image pair as the processing target (Step S206).

Next, the image acquiring function 11b similarly acquires the image data from the place of the comparison image (Step S207). Also in this case, when the acquisition of the comparison image data has failed due to a communication error, a file I/O error, or the like, the process returns to Step S204, and the loop processing is continued for the next pair in the list of the image pair as the processing target (Step S208). When the acquisition has been successful, the process proceeds to Step S209.

Next, the subtraction image acquiring function 11d acquires subtraction image data based on the first image data and the comparison image data selected by the processing step of Step S204 (Step S209). Specifically, the subtraction image acquiring function 11d invokes subtraction image calculation processing to achieve acquisition of the subtraction image. In this case, the subtraction image calculation processing may be executed by, other than being executed in the image processing apparatus 10, another apparatus on a cloud or the like. The subtraction image data refers to data of a result of subtracting, from a voxel value of each set of coordinates of the first image data, a voxel value of coordinates corresponding to the comparison image data associated through registration.

Further, the subtraction image is not required to be acquired in Step S209. The first image and the comparison image may be displayed on a display unit in a form of being comparable to each other (being arranged side by side, overlapping each other, or being switchable). Further, without performing display or subtraction image acquisition, the information of the image pair may only be saved or output (so that other image display apparatus or subtraction image generating apparatus can use the information).

Those processing steps are achieved by, for example, the processing circuit 11 invoking programs corresponding to the image acquiring function 11b and the subtraction image acquiring function 11d from the storage circuit 13 and executing the programs. After the subtraction image data is obtained for all of the pairs in the list of the image pair or display thereof or the like is performed, the flow proceeds to Step S210 so that the above-mentioned processing is ended.

In the description above, an example of a case in which both of contrast and non-contrast images are taken at a plurality of time points has been described. However, the embodiment of the present invention is not limited thereto, and the processing of generating an image pair may be performed for a more limited number of images as a target.

In the above-mentioned example, as the past image (comparison image), an image taken before occurrence of the invasion or the tumor is required, and as the present image (first image), an image taken after the invasion or the tumor has occurred is required. However, the past image may be absent in cases immediately after hospitalization or hospital transfer, for example. Further, in the contrast phase subtraction image generation method, a plurality of images having different contrast enhancement states are required to be obtained with an appropriate contrast agent being administered to the patient and presence or absence of this contrast effect being examined. However, in some cases, imaging using a contrast agent cannot be performed depending on the medical condition or constitution (allergy or the like) of the patient, and the contrast image is absent. How to cope with such a case is described in detail in the following.

As a specific example of such a case, a case in which, in FIG. 4A, only three images of the contrast images C3 and C1 and the non-contrast image N3 are present is described. When the first image data is the image C3, the priority of the image pair of the image C3 and an image other than the image C3 is, in a case in which the examination object is “visualization of invasion in spinal canal,” 1,202.0 for (C3-C1) and 1.0 for (C3-N3). Accordingly, the image processing apparatus 10 attempts to acquire the image C1 of the past examination as the comparison image, and when the image C1 cannot be acquired due to a communication error, a file I/O error, or the like, acquires the image N3 different in contrast enhancement state from the image C3 as the comparison image. Further, also when, in FIG. 4A, the image C1 of the past examination is absent and only two images of the contrast image C3 and the non-contrast image N3 are present, the image processing apparatus 10 acquires the image N3 different in contrast enhancement state from the image C3 as the comparison image.

Further, when the examination object is “visualization of liver tumor,” the priority becomes 1,202.0 for (C3-C1) and 10,000.0 for (C3-N3). Accordingly, the image processing apparatus 10 attempts to acquire the image N3 of an examination different in contrast enhancement state as the comparison image, and when the image N3 cannot be acquired due to a communication error, a file I/O error, or the like, acquires the image C1 of the past examination taken before the image C3 as the comparison image. Further, also when, in FIG. 4A, the image N3 of the examination different in contrast enhancement state is absent and only two images of the contrast image C3 and the past image C1 are present, the past image C1 taken before the contrast image C3 is acquired as the comparison image.

### Modification Example 1 of First Embodiment

In the above-mentioned first embodiment, the priority is calculated in accordance with the examination object, and an image pair having a high priority is selected. However, Modification Example 1 is not limited thereto. For example, the examination object may be fixed to “visualization of invasion in spinal canal,” and an image of an examination different in contrast enhancement state may be simply acquired when an image of a past examination is absent. Further, the examination object may be fixed to “visualization of liver tumor,” and an image of a past examination may be simply acquired when an image of an examination different in contrast enhancement state is absent.

Further, processing corresponding to the examination object is not always required. For example, regardless of the examination object, an examination image of a past examination may be acquired in priority as the comparison image, and when the past examination image is absent, an examination image different in contrast enhancement state in the target examination may be acquired as the comparison image. As another example, regardless of the examination object, an examination image different in contrast enhancement state in the target examination may be acquired in priority as the comparison image, and when the examination image different in contrast enhancement state is absent, an examination image of a past examination may be acquired as the comparison image.

As described above, through use of the image processing apparatus according to the first embodiment, the temporal subtraction image generation processing and the contrast phase subtraction image generation processing can be appropriately switched. In this manner, even when a past image comparable to the image of the target examination is absent such as immediately after hospitalization or hospital transfer, or even when the contrast image is absent because of poor health or allergy, a subtraction image for visualizing a lesion can be generated without effort. Further, the priority is given based on the examination date/time and the contrast enhancement state in accordance with the examination object, and thus a subtraction method more suitable for visualization of the lesion can be selected. In this manner, an appropriate image pair for visualization of the lesion can be selected without requiring a troublesome effort of the user, and the subtraction image can be generated through use of this image pair.

### Modification Example 2 of First Embodiment

In the first embodiment, the date/time difference and the contrast phase are used as parameters as the selection condition at the time of generating the image pair. For example, when the examination object is “visualization of invasion in spinal canal” or “visualization of liver tumor,” it is effective to use those items as parameters. However, the priority may be set in view of, for example, a degree of coincidence of an imaging site depending on the examination object.

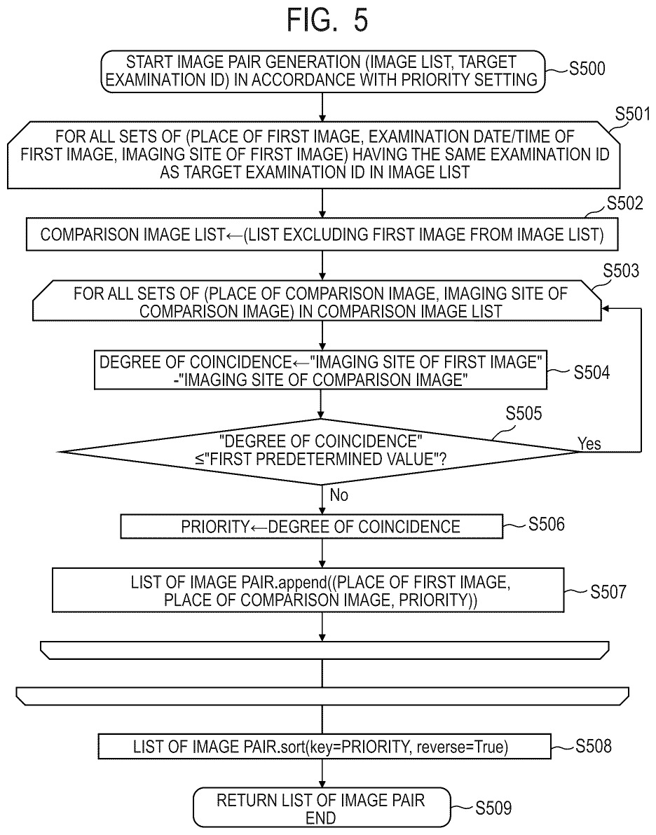

Now, regarding Modification Example 2, sub-steps of image pair generation to be executed in Step S203 are described with reference to the flow chart of FIG. 5 from Step S500 to Step S509. In Step S501, specifically, each of all images having the same examination ID as the target examination ID in the image list obtained in Step S201 (that is, images of the target examination) is the first image. In addition, through use of information (place of image, imaging site) of the first image, loop processing of from Step S502 to Step S507 is executed by the comparison image acquiring function 11c.

First, a list excluding the first image from the image list is acquired as a comparison image list (Step S502). Next, for each of all images in the comparison image list as a candidate for the comparison image, information (for example, place of the image, imaging site) of the comparison image is subjected to loop processing of from Step S504 to Step S507 (Step S503). First, the imaging site included in the first image and the imaging site included in the comparison image are compared to each other so that the degree of coincidence of both of the imaging sites is checked. When the imaging sites are the same, the degree of coincidence is the maximum, and when the imaging sites are completely different from each other, the degree of coincidence is 0. Further, when the imaging sites have different sizes or partially overlap each other, the degree of coincidence is determined in accordance with the range of the overlapping part. When the degree of coincidence is a first predetermined value or smaller, the images are determined as being inappropriate for forming an image pair, and when the degree of coincidence is larger than the first predetermined value, it is determined that the images can be used as an image pair so that the subtraction image can be calculated (Step S505).

When the subtraction image can be calculated, the degree of coincidence is set as the priority in Step S506. Information of a combination of the place of the first image, the place of the comparison image, and the priority which have been acquired by the above-mentioned processing steps is appended to the list of the image pair (Step S507). After appending to the list, the flow returns to Step S503, and the priority is obtained for each image pair based on the comparison image list obtained in Step S502 so that the image pair is appended to the list as appropriate.

After the calculation of the priority of all image pairs based on the comparison image list obtained in Step S502 is ended, the flow returns to Step S501, and processing of from Step S502 to Step S507 is performed for a new first image. After the processing of obtaining the priority of the image pair is ended for all images having the same examination ID as the target examination ID and thus the process exits from a double loop, the list of the image pair is sorted in descending order through use of the priority as a key (Step S508). With the processing of from Step S500 to Step S509 described above, a list of image pairs arranged in descending order of priority is acquired.

As described above, the image processing apparatus according to the first embodiment includes the image acquiring function 11b serving as a first image acquiring unit, and the comparison image acquiring function 11c serving as a second image acquiring unit. The image acquiring function 11b acquires first image data which is image data obtained by imaging a subject by a first examination. Further, the comparison image acquiring function 11c acquires comparison image data for performing comparison to the first image data. Further, the comparison image acquiring function 11c calculates priority of a combination (in the embodiment, an image pair) including the first image data and a candidate for the comparison image data, based on an examination date/time of the first examination and a contrast enhancement state of the first image data. The comparison image acquiring function 11c further acquires the comparison image data to be used for generation of a subtraction image, in accordance with the calculated priority. Further, the image processing apparatus according to the present disclosure can also execute the following image processing method. That is, the image acquiring function 11b executes a first image acquiring step of acquiring first image data, and the comparison image acquiring function 11c executes a comparison image acquiring step of acquiring comparison image data.

As described in Modification Example 1, when the comparison image data is determined, there is also assumed a case in which the past image or the contrast image is absent. In such a case, the image pair for which the priority is calculated based on the examination date/time of the first examination and the contrast enhancement state of the first image data cannot be formed. In such a case, a combination of images for which the priority can be calculated can be formed when the formation is based on any one of the examination date/time of the first examination or the contrast enhancement state of the first image data. In such a case, for example, the comparison image acquiring function 11c skips any of Step S304 or Step S305 of FIG. 3, and determines the comparison image data based on a pair for which the priority can be calculated. More specifically, the comparison image acquiring function 11c attempts to acquire, as the comparison image data, second image data which is image data obtained by imaging the subject by a second examination which is a past examination performed before the first examination. Then, when the second image data cannot be acquired, third image data which is image data different in contrast enhancement state from the first image data is acquired as the comparison image data. As another example, the comparison image acquiring function 11c can also attempt to acquire, as the comparison image data, second image data which is image data obtained by imaging the subject by a second examination performed under a contrast enhancement state different from that of the first examination. In this mode, when the second image data cannot be acquired, the comparison image acquiring function 11c acquires, as the comparison image data, third image data which is image data obtained by imaging the subject by a third examination which is a past examination performed before the first examination.

The first image data and the comparison image data forming the image pair are acquired by the above-mentioned image processing apparatus. However, as described above, the subtraction image acquiring function 11d serving as a subtraction image acquiring unit can be configured separately from the configuration including the image acquiring function and the comparison image acquiring function. In addition, this subtraction image acquiring function calculates the subtraction image data through subtraction operation between the first image data and the comparison image data forming the combination of images, to thereby acquire the subtraction image data.

Further, the image processing apparatus according to the present disclosure further includes the priority setting function 11e serving as a priority setting unit configured to set the priority. The priority setting function 11e compares a date/time difference between a first time/date at which the first examination is performed and a second time/date at which an examination is performed to obtain the comparison image data to a predetermined value determined in advance. In the above-mentioned embodiment, for example, one week or two days are set as the predetermined value. In addition, the priority of a combination including the first image data and the comparison image data whose date/time difference is larger than this predetermined value is set to be higher than the priority of a combination including the first image data and the comparison image data whose date/time difference is smaller than this predetermined value.

The priority setting function 11e further changes the setting of the priority between a case of the combination including the comparison image data whose date/time difference between the first date/time and the second date/time is larger than the predetermined value and a case of the combination including the comparison image data whose date/time difference is equal to or smaller than the predetermined value. Specifically, when the date/time difference is larger than the predetermined value, the priority of an image pair including the comparison image data having a small difference in contrast enhancement state from the first image data is set to be higher than the priority of an image pair including the comparison image data having a large difference in contrast enhancement state. Further, when the date/time difference is equal to or smaller than the predetermined value, the priority of the image pair including the comparison image data having a large difference in contrast enhancement state from the first image data is set to be higher than the priority of the image pair including the comparison image data having a small difference in contrast enhancement state. In addition, this priority setting unit can set those priorities based on the examination object. Further, in this case, when the comparison image data is selected, the image pair is generated and the priority is calculated at this time, but there may be employed a method in which the priority may be calculated for all of the image pairs in advance and the image pair is selected in accordance with this priority.

Further, in the present disclosure, the image acquiring function 11b acquires a candidate for the first image data which is the image data obtained by imaging the subject by the first examination, and the comparison image acquiring function 11c acquires a candidate for the comparison image data for performing comparison to the first image data. In the embodiment, the priority setting function 11e can also serve as a priority acquiring unit to acquire the priority with respect to a combination including each of those candidates for the first image data and each of those candidates for the comparison image data. When the priority is acquired for such a combination, the image acquiring function 11b can acquire, for example, the first image data to be used for generation of the subtraction image from among the candidates for the first image data, in accordance with the acquired priority. Further, the comparison image acquiring function 11c can acquire, for example, the comparison image data to be used for generation of the subtraction image from among the candidates for the comparison image data, in accordance with the acquired priority.

As described above, the present disclosure can determine the priority of generating the image pair even based on the degree of coincidence of the imaging site or the size of the overlapping region. In Modification Example 2, an appropriate image pair for visualizing the lesion can be selected based on the level of the degree of coincidence without requiring a troublesome effort of the user, and the subtraction image can be generated through use of this image pair. Further, the priority may be determined through use of the date/time difference and the degree of coincidence used in Modification Example 2. As another example, in the first embodiment, for example, when an appropriate image pair cannot be obtained due to shortage of images or the like, the parameter for determining the priority may be changed to the degree of coincidence described in Modification Example 2 so that the comparison image is retrieved. Further, when the image data includes other information, for example, other than the image pair, the degree of coincidence of pieces of supplementary information can be used, or those pieces of supplementary information can be targets to which the above-mentioned parameters are caused to refer. Accordingly, as the priority at the time of obtaining an image pair formed of the first image data and the second image data, the priority determined for a combination including the first image data and the second image data may be used.

### Second Embodiment

In the above-mentioned first embodiment, as the processing step of Step S209, a subtraction image between the first image data and the comparison image data acquired in the previous steps is acquired. In this processing of acquiring the subtraction image, a calculating method may be changed based on a relationship between the first image data and the comparison image data, and this case may also be included in one embodiment of the present invention. An example of this case is described below as a second embodiment of the present disclosure.

The overall configuration of the image processing apparatus of the second embodiment is similar to the overall configuration of the first embodiment described with reference to FIG. 1, and hence detailed description thereof is omitted here. Further, the flow chart of the processing to be executed by the image processing apparatus of the second embodiment is similar to the flow chart of the first embodiment described with reference to FIG. 2. However, the processing of acquiring the subtraction image data executed as Step S209 is different from that of the first embodiment, and hence this processing of acquiring the subtraction image data is described in detail in the following.

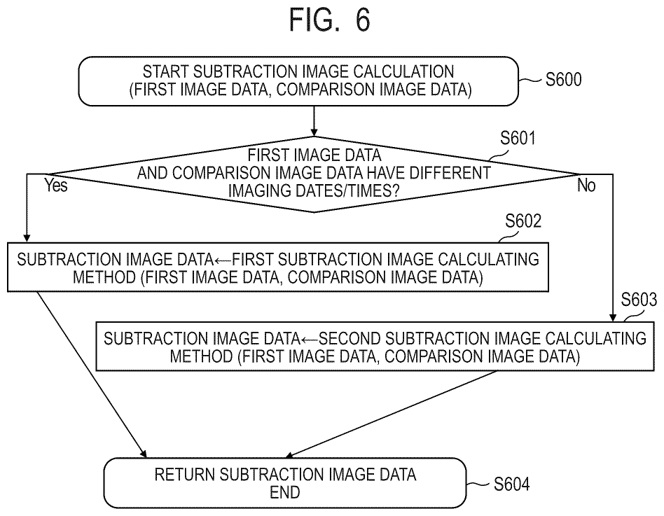

FIG. 6 is a flow chart for illustrating in detail the processing performed in Step S209 in the second embodiment. As illustrated in FIG. 6, the image processing apparatus according to the second embodiment switches the method of calculating the subtraction image data depending on, for example, whether or not the first image data and the comparison image data have different imaging dates/times (Step S601). Specifically, in Step S601, when the first image data and the comparison image data have different imaging dates/times, the process proceeds to Step S602, and otherwise the process proceeds to Step S603.

When the imaging dates/times are different, the posture or the body shape of the patient is often different between the first image and the comparison image, and it is desired to perform appropriate comparison operation for those images. Accordingly, the subtraction image data is calculated by a first subtraction image calculating method (Step S602).

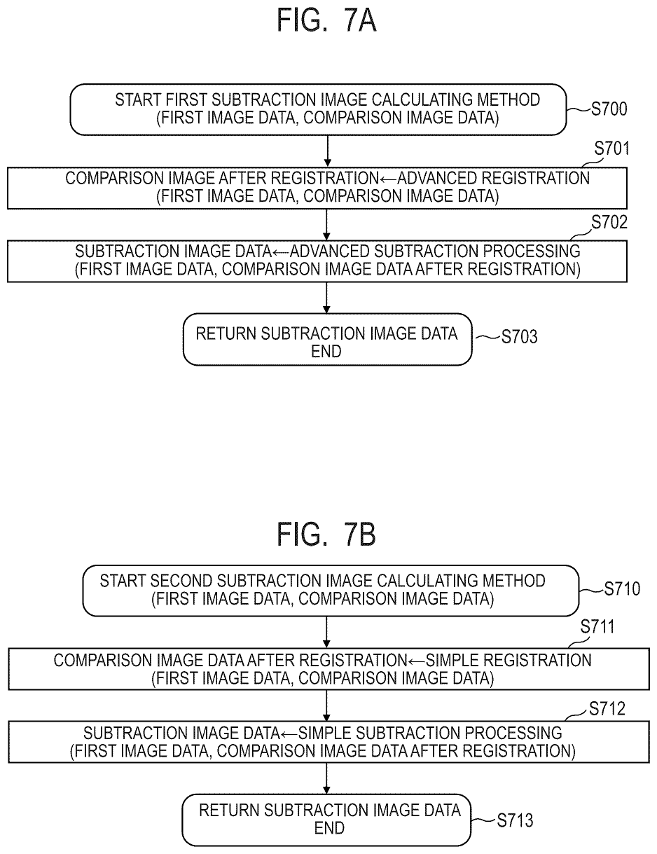

FIG. 7A is a flow chart of the specific processing performed in Step S602.

In the processing illustrated in FIG. 7A, the first image data and the comparison image data are subjected to registration by a method that is robust to the change of the posture or the body shape of the subject between the images. In the example illustrated in FIG. 7A, in Step S701, advanced registration such as a large deformation diffeomorphic metric mapping (LDDMM) method is performed. Then, the subtraction image data is calculated by advanced subtraction processing that can calculate the subtraction so as to be robust to the residual of deformable registration or the like, such as an adaptive voxel matching method (Step S702).

Meanwhile, when the imaging dates/times are the same, the difference in posture or body shape of the patient is slight between the first image and the comparison image, and the difference is often only the contrast enhancement state, mainly. Accordingly, the subtraction image is calculated by a second subtraction image calculating method, which is simpler than the first subtraction image calculating method (Step S603). Specifically, as illustrated in FIG. 7B, simple registration such as correction of only translation or rotation between the two images is performed (Step S711). Then, after the registration is ended, the subtraction image data is calculated through simple subtraction processing such as simple subtraction between pixel values of coordinates corresponding to both of the images (Step S712).

In this case, after the image pair is generated, the method of calculating the subtraction image is changed in consideration of occurrence of difference in posture or body shape of the patient depending on whether the imaging dates/times are different from each other. However, regardless of the imaging date/time, the subtraction image calculating method may be changed in consideration of, for example, difference in contrast enhancement state between the first image data and the comparison image data. Specifically, for example, when a subtraction image between images having different contrast enhancement states is calculated, it is desired to exclude organs, blood vessels, and the like that greatly change in concentration value depending on the presence or absence of the contrast enhancement, and use a subtraction image calculating method using registration processing or subtraction processing for other sites.

As described above, in the second embodiment, the subtraction image acquiring function 11d can execute at least one of the first subtraction image calculating method (Step S602) or the second subtraction image calculating method (Step S603) which are calculating methods different from each other. The subtraction image acquiring function 11d can select any of those calculating methods based on the comparison image data, and can calculate the subtraction image data through use of the selected subtraction image calculating method. In more detail, the first subtraction image calculating method and the second subtraction image calculating method execute registration processing having characteristics different from each other. In addition, the subtraction image data is calculated based on a result of the registration between the first image data and the comparison image data performed by using the registration processing.

According to the method described above, the subtraction image calculating method is changed based on the comparison image data, and thus there is provided an effect of allowing appropriate comparison operation to be performed between the first image and the comparison image. Further, the second embodiment may be applied so that, when the image pair is formed with reference to the priority that is based on the degree of coincidence described in Modification Example 2 of the first embodiment, the registration method is changed between a case in which the degree of coincidence is low and a case in which the degree of coincidence is high.

### Third Embodiment

Another embodiment according to the present disclosure includes a mode of calculating subtraction images from first image data and a plurality of pieces of comparison image data, and obtaining an evaluation value for each calculation process, thereby obtaining the priority of the image pair based on this evaluation value. In this case, a subtraction image having the best evaluation value becomes the subtraction image that is finally presented to the user. According to a third embodiment, a subtraction image that more appropriately visualizes the lesion can be acquired based on results of the processing of calculating actual subtraction images between the first image data and the plurality of pieces of comparison image data.

At this time, the plurality of pieces of comparison image data may be all pieces of image data other than the first image data obtained by imaging the same subject as that of the first image data, or may be a part of those pieces of image data. In order to select the part of the pieces of image data, the method of calculating the priority described in the first embodiment may be used so that the processing of calculating the subtraction image is executed for the image data having priority of a predetermined value or more.

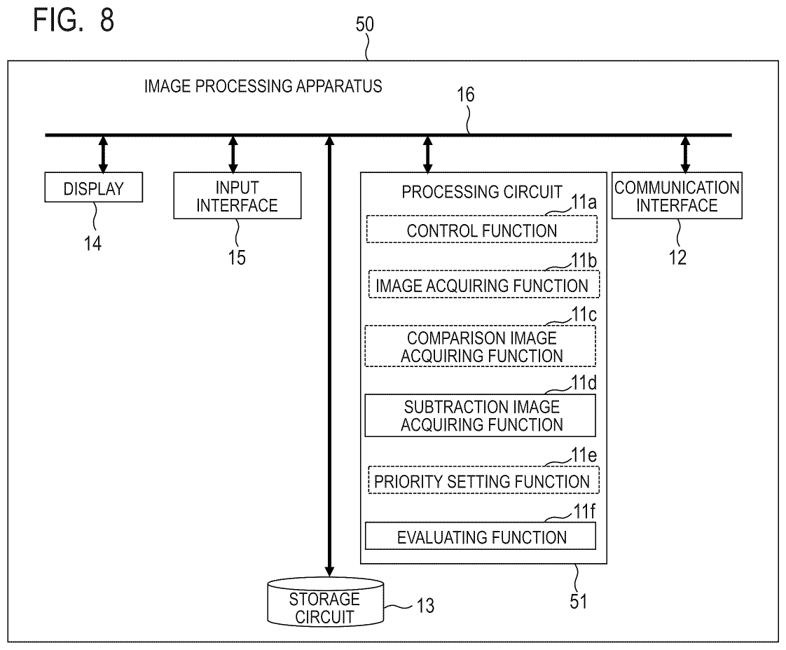

FIG. 8 shows an overall configuration of an image processing apparatus 50 according to the third embodiment. A processing circuit 51 in the third embodiment is different from the processing circuit 11 in each of the first embodiment and the second embodiment in the processing content performed by the subtraction image acquiring function 11d. Further, an evaluating function 11f is added. Specifically, the subtraction image acquiring function 11d in the third embodiment acquires a subtraction image with evaluation value to be described later so as to acquire subtraction image data and an evaluation value used when the subtraction image data is calculated. In addition, the evaluating function 11f selects the subtraction image data having the highest evaluation value. Now, those points are mainly described.

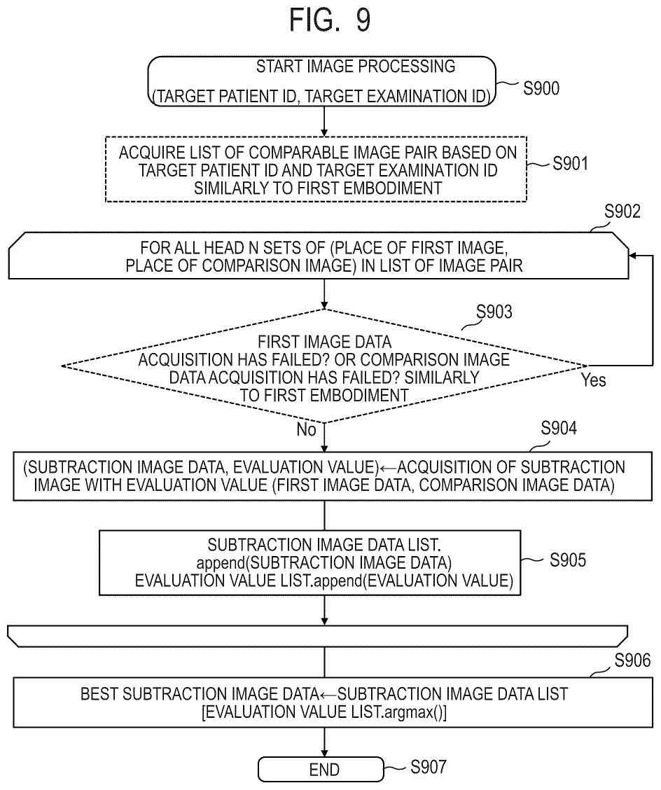

FIG. 9 is a flow chart for illustrating all steps of the image processing apparatus 50 according to the third embodiment. In this case, in Step S901 indicated by the dotted-line frame of FIG. 9, the processing steps of from Step S201 to Step S203 of FIG. 2 are executed, and, in Step S903, the processing steps of from Step S205 to Step S208 of FIG. 2 are executed. Further, a processing step of Step S904 illustrated in FIG. 9 is achieved by, for example, the processing circuit 51 invoking a program corresponding to the subtraction image acquiring function 11d from the storage circuit 13 and executing the program. Further, processing steps of Step S902 and from Step S905 to Step S906 illustrated in FIG. 9 are achieved by, for example, the processing circuit 51 invoking a program corresponding to the evaluating function 11f from the storage circuit 13 and executing the program.

In Step S902, the image processing apparatus 50 repeatedly executes the subsequent processing steps of from Step S903 to Step S905 as loop processing for information (place of first image, place of comparison image) of head N image pairs in the list of the image pair. That is, the loop processing is performed in order from the information of the image pair having higher priority. In this case, when N is increased, the processing of calculating the subtraction image is actually performed for a large number of image pairs and it takes long calculation time, and hence N is desired to be a value of from about ½ to about ¼ of the length of the list.

In Step S904, similarly to Step S208 in the first embodiment, when the subtraction image acquiring function 11d has succeeded in acquisition of the comparison image data, the subtraction image data between the first image data and the comparison image data is acquired. However, the image processing apparatus 50 according to the third embodiment is different from the first embodiment in that the subtraction image data is acquired with an evaluation value (Step S904).

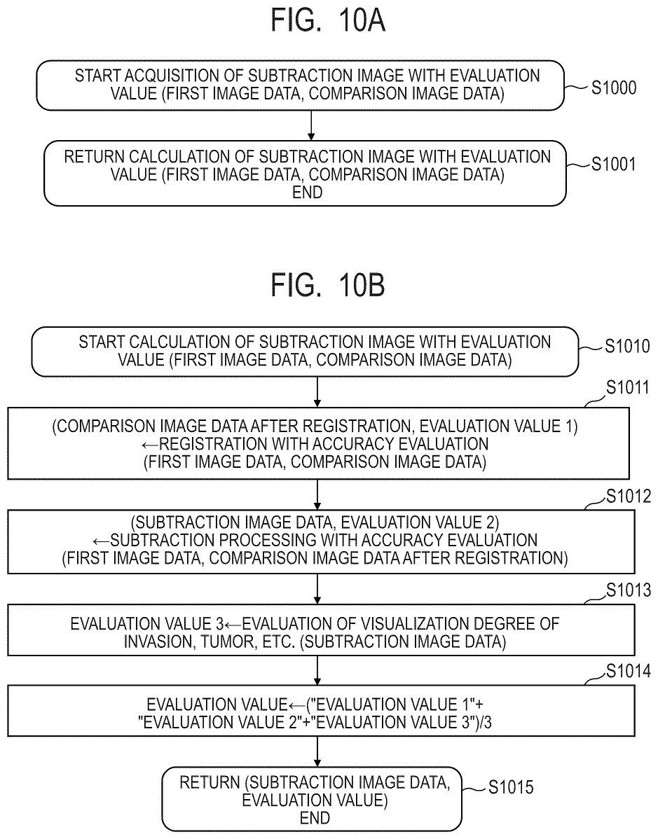

Specifically, in Step S904, as illustrated in FIG. 10A, the subtraction image acquiring function 11d achieves acquisition of the subtraction image by invoking processing of calculating a subtraction image with evaluation value (Step S1001). FIG. 10A is a flow chart of from Step S1000 to Step S1001 for illustrating details of sub-processing steps performed in Step S904. In this case, the processing of calculating the subtraction image with evaluation value may be executed by, other than being executed in the image processing apparatus 50, another apparatus on a cloud or the like.

The evaluation value used at the time of subtraction image calculation is, for example, a representative value calculated from the following three evaluation values. Now, processing of calculating the evaluation value to be executed in Step S1011 to Step S1014 is described with reference to the flow chart of FIG. 10B for illustrating the processing of calculating the evaluation value.

An evaluation value 1 of FIG. 10B is an evaluation value calculated in Step S1011, and is an evaluation value relating to the registration processing between the first image data and the comparison image data. For example, a good evaluation value is obtained when there is no local distortion in a spatial correspondence between images, which is a result of the registration processing, or when convergence of transition of similarity is high in iteration processing of maximizing the similarity between the images at the time of registration.

In general, when the registration between images of the same patient is appropriately performed, local distortion rarely occurs in the correspondence between the images. Thus, the evaluation of the local distortion can be used to perform evaluation of whether or not the registration has been appropriately performed. Further, in general, when an image feature that becomes a clue of the registration between the images is present, the image similarity in the iteration processing exhibits a high convergence, and registration having high reliability is performed. Meanwhile, when the image feature is poor, the convergence of the image similarity is low, and the reliability of the registration is low. Thus, the evaluation of the convergence can be used to perform the evaluation of the reliability of the registration.

An evaluation value 2 of FIG. 10B is an evaluation value calculated in Step S1012, and is an evaluation value relating to subtraction processing. For example, a better evaluation value is obtained as the calculated subtraction image has smaller noise caused by a partial volume effect or the like. The amount of noise in the subtraction image can be calculated through use of a known method such as machine learning or spatial frequency analysis.

An evaluation value 3 of FIG. 10B is an evaluation value calculated in Step S1013, and is an evaluation value indicating how clear the subtraction image data visualizes the lesion. Specifically, for a part having a large subtraction value on the calculated subtraction image, whether or not an image feature of a first image or a comparison image corresponding to this part seems to be a lesion is determined. A better evaluation value is obtained when it is determined that this part seems to be a lesion. The determination of whether or not this part of the first image or the comparison image seems to be a lesion can be executed through use of a known method such as machine learning or spatial autocorrelation.

As a final evaluation value used at the time of subtraction image calculation, the evaluation value can be calculated by calculating, for example, an average value as a value obtained by integrating the above-mentioned three evaluation values (Step S1014). Further, the evaluation value can be calculated through use of, other than the average value, the maximum value, the minimum value, or other values, or through use of multiplication by each evaluation value. With the above-mentioned processing steps, it is possible to calculate an evaluation value evaluating whether or not a subtraction image in which registration between images is appropriately executed, unnecessary noise that hinders the visualization of the lesion is small, and a lesion part which is an object of the visualization can be easily recognized has been generated. With the processing steps of from Step S1010 to Step S1015 described above, subtraction data with evaluation value is calculated.

The subtraction image data and the evaluation value calculated as described above are appended to a subtraction image data list and an evaluation value list, respectively (Step S905). After the above-mentioned calculation processing is ended for all of the image pairs, the evaluating function 11f calculates an index having the maximum evaluation value in the evaluation value list, and acquires data in the subtraction image data list corresponding to this index as the best subtraction image data having the maximum evaluation value (Step S906).

The image processing apparatus according to the third embodiment includes the evaluating function 11f (evaluating unit) in addition to, for example, the above-mentioned image acquiring function 11b (image acquiring unit), comparison image acquiring function 11c (comparison image acquiring unit), and subtraction image acquiring function 11d (subtraction image acquiring unit). The image acquiring function 11b acquires first image data which is image data obtained by imaging a subject by a first examination. The comparison image acquiring function 11c acquires, as first comparison image data, image data obtained by imaging the subject in a contrast condition different from a contrast condition of the first image data in the first examination and acquires, as second comparison image data, image data obtained by imaging the subject by a second examination performed at an examination date different from an examination date of the first examination. The subtraction image acquiring function 11d acquires first subtraction image data representing subtraction between the first image data and the first comparison image data and second subtraction image data representing subtraction between the first image data and the second comparison image data. The evaluating function 11f performs evaluation of each of the first subtraction image data and the second subtraction image data. The subtraction image acquiring function 11d selects subtraction image data having a better result of the evaluation from the first subtraction image data and the second subtraction image data.

As described above, through use of the image processing apparatus according to the third embodiment, a subtraction image suitable for visualization of a lesion can be selectively generated from among a plurality of subtraction images. In this manner, a subtraction image for allowing visualization of a lesion can be generated without requiring troublesome effort of the user.

### Fourth Embodiment

Another embodiment according to the present disclosure includes a mode of requesting execution confirmation from the user through use of the input interface 15 at the time of calculating the subtraction image, and displaying the calculated subtraction image on the display 14. For example, when the image processing apparatus 10 is implemented as an application to be executed on a PC, a workstation, or the like, the above-mentioned mode is useful for improvement of the ease of use of this application.

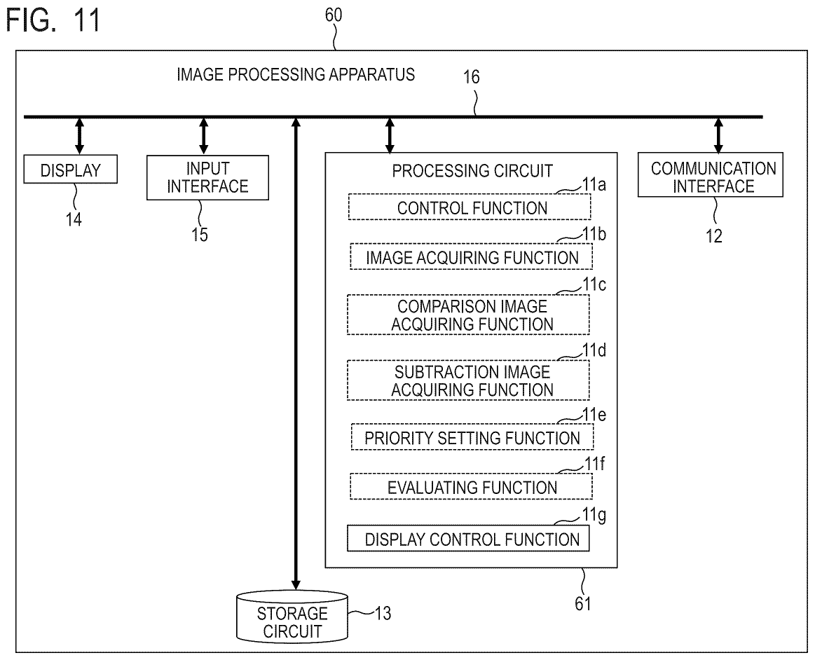

FIG. 11 shows an overall configuration of an image processing apparatus 60 according to a fourth embodiment. The fourth embodiment has a display control function 11g added thereto as compared to the first embodiment, the second embodiment, and the third embodiment.

Specifically, the display control function 11g in the fourth embodiment performs user confirmation at the time of subtraction image calculation and display of the subtraction image on the display 14. Those points are hereinafter mainly described.

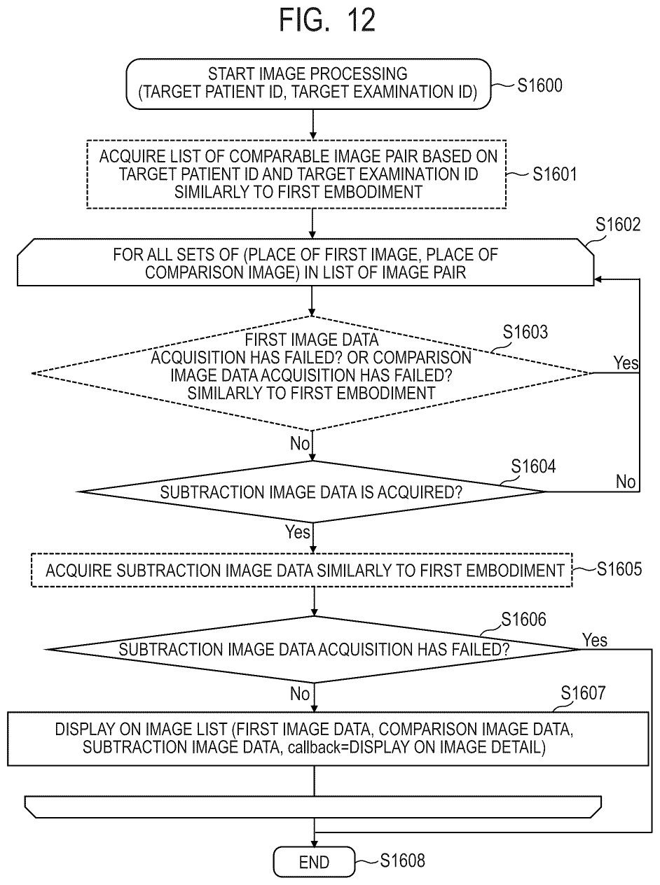

FIG. 12 is a flow chart for illustrating all steps of the image processing apparatus 60 according to the fourth embodiment. In this case, in Step S1601 indicated by the dotted-line frame of FIG. 11, the processing steps of from Step S201 to Step S203 in the first embodiment are executed, and, in Step S1603, the processing steps of from Step S205 to Step S208 are executed. Further, in Step S1605, the processing step of Step S209 in the first embodiment is executed. Further, processing steps of Step S1604, Step S1606, and Step S1607 illustrated in FIG. 11 are achieved by, for example, a processing circuit 61 invoking a program corresponding to the display control function 11g from the storage circuit 13 and executing the program.

In Step S1604, similarly to Step S208 in the first embodiment, when the comparison image data acquisition has been successful in the processing step of Step S1603, the display control function 11g determines whether or not to acquire the subtraction image data. However, the fourth embodiment is different from the first embodiment in that the processing is changed based on the execution confirmation performed by the user (Step S1604).

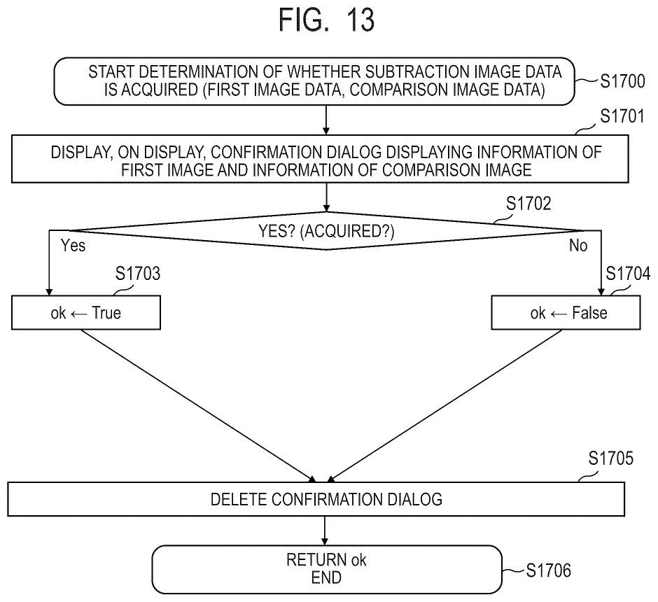

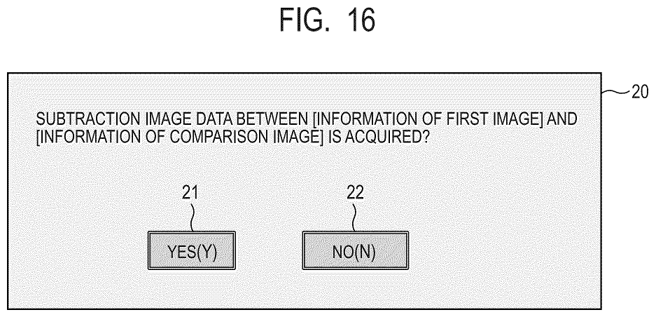

The processing performed in Step S1604 is specifically described with reference to the flow chart of sub-processing steps illustrated in FIG. 13. In Step S1604, as illustrated in FIG. 13, the display control function 11g invokes processing of determining whether or not to acquire the subtraction image data (Step S1700). In this processing, first, for example, as illustrated in FIG. 16, a confirmation dialog 20 displaying information of the first image and information of the comparison image is displayed on the display 14 (Step S1701). As the information to be displayed, for example, the examination date/time, a series number, or the like is displayed. In addition to those pieces of information, for example, a tomographic image or a projection image of each of the first image and the comparison image may be displayed.

Next, it is determined which of a “YES” button 21 (acquired) or a “NO” button 22 is pressed by the user via the input interface 15 (Step S1702). The determination result is stored in a Boolean variable ok (Step S1703, Step S1704), and after the confirmation dialog 20 is deleted (Step S1705), the determination result is returned (Step S1706).

When the determination result is No (not acquired), the next candidate in the list of the image pair is extracted and the iteration processing is performed (Step S1602). In this case, instead of performing the iteration processing, the processing may be ended. When the determination result is Yes (acquired), similarly to the first embodiment, the subtraction image data is acquired (Step S1605). When the subtraction image data acquisition has failed, the processing is directly ended. At this time, the display control function 11g may display some error dialog on the display 14 (Step S1606).

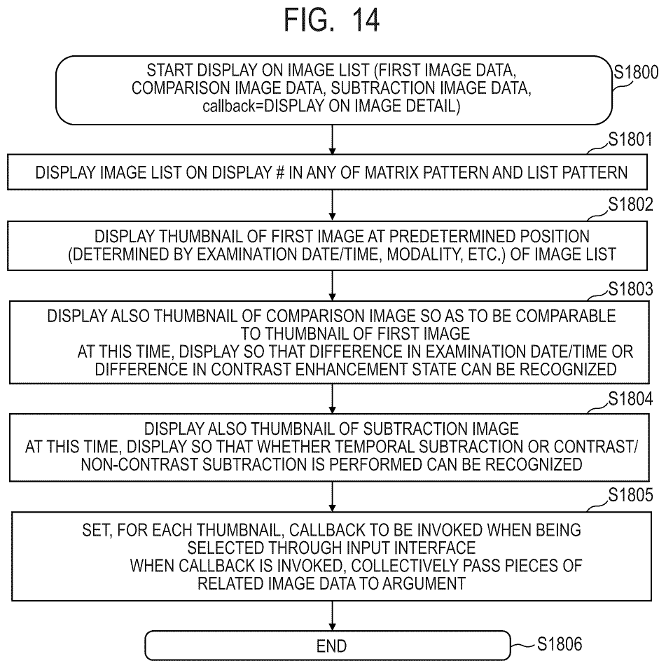

When the subtraction image data acquisition has been successful, for example, the display control function 11g displays the first image, the comparison image, and the subtraction image on an image list screen (Step S1607). The processing to be performed in Step S1607 is specifically described with reference to the flow chart of sub-processing steps illustrated in FIG. 14. Specifically, as illustrated in FIG. 14, together with the first image data, the comparison image data, and the subtraction image data, a callback for displaying given image data on an image detail screen is designated, and processing of displaying on the image list is invoked (Step S1800).

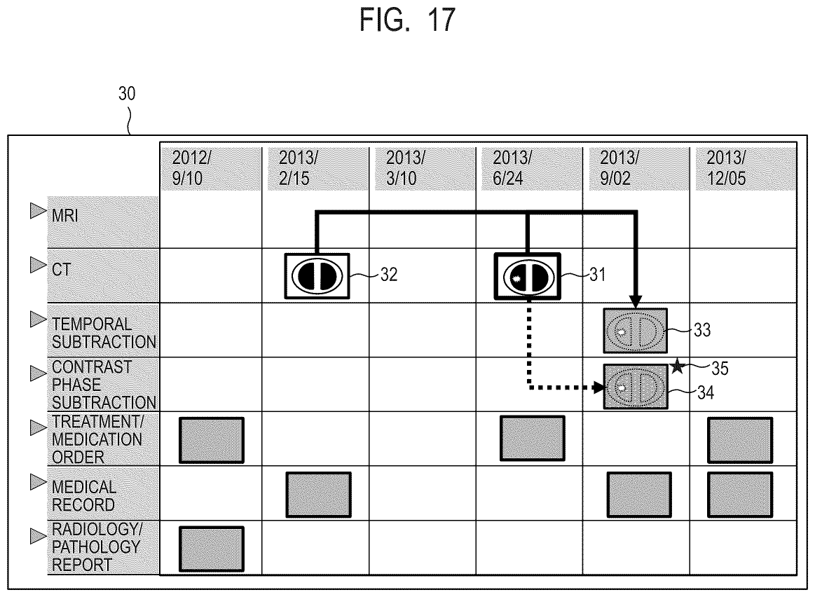

In this processing, for example, an image list screen 30 as illustrated in FIG. 17 is displayed on the display 14 (Step S1801). The image list screen 30 is not limited to a mode of displaying thumbnails in a matrix pattern, and may be in a mode of displaying thumbnails in a list pattern. Next, a thumbnail 31 of the first image is displayed at a predetermined position determined based on the examination date/time of the first image, the modality, and the like (Step S1802). Next, a thumbnail 32 of the comparison image is also displayed so as to be comparable to the thumbnail of the first image (Step S1803). At this time, the thumbnail 32 is displayed so that the difference in examination date/time and the difference in contrast enhancement state can be recognized. For example, text information on the difference in examination date/time (number of days), or a mark indicating inside or outside of the range of the date/time difference boundary value of the examination date/time may be displayed adjacent to the thumbnail or in superimposition thereto. As another example, text information or a mark indicating presence or absence of the difference in contrast enhancement state or the magnitude of the difference may be displayed adjacent to the thumbnail or in superimposition thereto.

Moreover, a thumbnail 33 (in the case of temporal subtraction) or a thumbnail 34 (in the case of contrast phase subtraction) of the subtraction image is also displayed (Step S1804). At this time, for example, when the examination aims to visualize the invasion in the spinal canal, for example, the contrast phase subtraction image having low priority is marked with a star-shaped icon 35 of FIG. 17 so that the contrast phase subtraction image can be displayed while being distinguished from the temporal subtraction image, thereby calling user's attention (Step S1804). Conversely, the temporal subtraction image having high priority may be marked so that the temporal subtraction image can be distinguished from another temporal subtraction image or contrast phase subtraction image having low priority. Further, callback to be invoked when being selected through the input interface 15 is set for each thumbnail (Step S1805). When this callback is invoked, related pieces of image data are collectively passed to the argument of the callback (Step S1806).

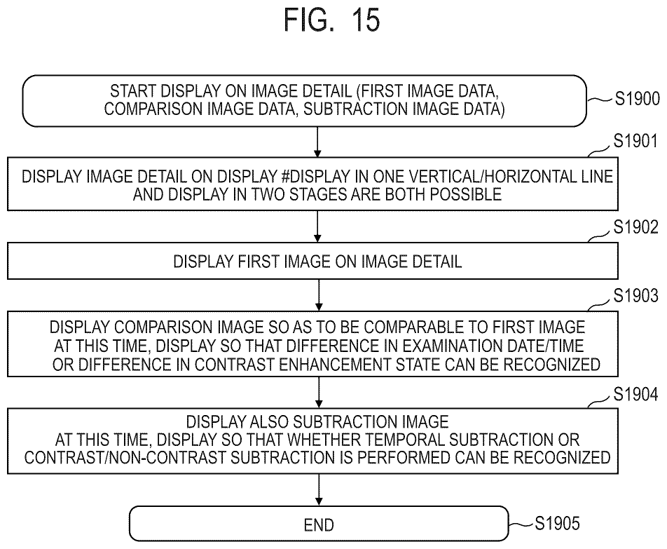

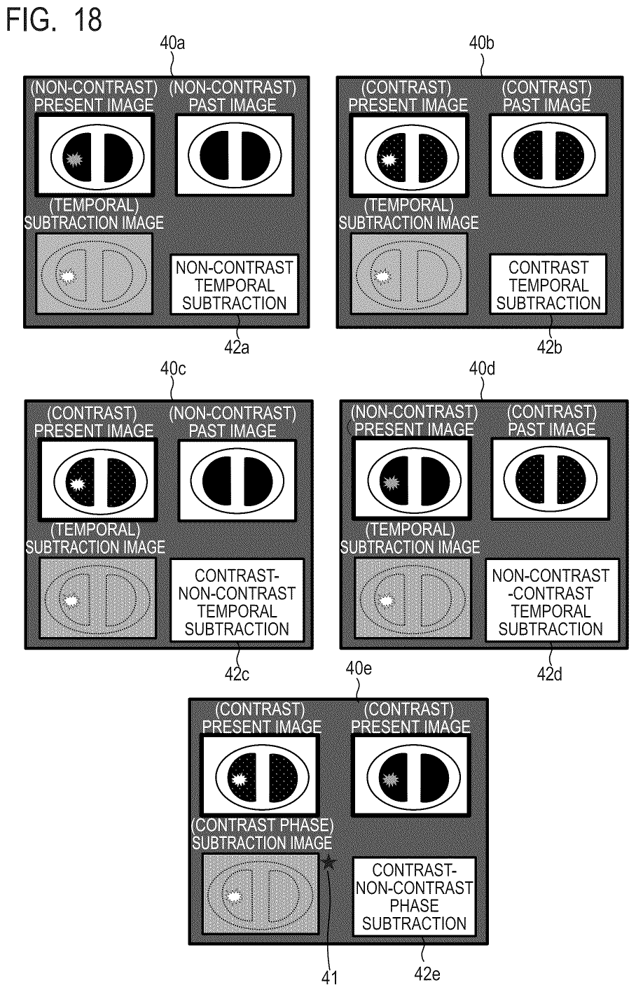

FIG. 15 shows a flow chart of from Step S1900 to Step S1905 for callback processing of displaying each image on the image detail screen. In the callback processing, first, the display control function 11g displays, for example, an image detail screen (any one of 40a, 40b, 40c, 40d, or 40e) as illustrated in FIG. 18 on the display 14 (Step S1901). The image detail screen is not limited to a mode of displaying images in two vertical and horizontal stages, but may be in a mode of displaying images in one vertical or horizontal line.

Next, the first image (present image) is displayed on the image detail screen (Step S1902). Next, the comparison image (past image) is displayed so as to be comparable to the first image (Step S1903). At this time, for example, label indication of the present image or the past image or label indication of contrast or non-contrast is performed above each image. Thus, the image is displayed so that the difference in examination date/time and the difference in contrast enhancement state thereof can be recognized. Moreover, the subtraction image is also displayed on the image detail screen (Step S1904). At this time, for example, as label indications of from an image detail screen 42a to an image detail screen 42e of FIG. 18, the image is displayed so that whether the image is a temporal subtraction image or a contrast phase subtraction image can be recognized.

Further, for example, when the examination aims to visualize the invasion in the spinal canal, for example, the contrast phase subtraction image having low priority may be marked with a star-shaped icon 41 of FIG. 18 so that the contrast phase subtraction image can be displayed while being distinguished from the temporal subtraction image. Conversely, the temporal subtraction image having the highest priority may be marked so that the temporal subtraction image can be distinguished from another temporal subtraction image or contrast phase subtraction image.

Description has been given in Step S1607 of FIG. 12 that, first, the thumbnail is displayed on the image list screen, and after selection through the input interface 15, the image detail screen is displayed. However, the mode of the fourth embodiment is not limited to the mode exemplified here, and the image detail screen may be directly displayed at a time point of Step S1607. With the processing steps of from Step S1600 to Step S1608 described above, a desired subtraction image can be appropriately displayed.

As described above, the image processing apparatus according to the fourth embodiment further includes the display control function 11g (display control unit) configured to perform processing of displaying the image pair on a display unit. This display control function 11g can be configured to cause the display unit to display a confirmation screen (FIG. 16) for confirming whether or not to acquire the subtraction image data. Further, the display control function 11g can also be configured to cause the display unit to display the first image data and the comparison image data so as to be comparable to each other (FIG. 17). Moreover, the display control function 11g can also be configured to cause the display unit to display the first image data, the comparison image data, and the subtraction image data so as to be comparable to each other (FIG. 18). In addition, the display control function 11g can also be configured to cause the display unit to display information corresponding to the priority (star-shaped icon 35 of FIG. 17).

As described above, through use of the image processing apparatus according to the fourth embodiment, whether or not each of various subtraction images is suitable for the examination object, or the degree of adaption to the examination object can be presented to the user. In this manner, the ease of use of the application can be improved, and the subtraction image for visualizing the lesion can be generated without requiring a troublesome effort of the user.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-081469, filed May 17, 2023, which is hereby incorporated by reference herein in its entirety.

What is claimed is:

1. An image processing apparatus comprising:
a first image acquiring unit configured to acquire first image data which is image data obtained by imaging a subject by a first examination; and
a comparison image acquiring unit configured to acquire comparison image data for performing comparison to the first image data, the comparison image acquiring unit being configured to acquire the comparison image data in accordance with priority with respect to a combination including the first image data and a candidate for the comparison image data.

2. The image processing apparatus according to claim 1, wherein the priority is configured to be calculated based on an examination date/time of the first examination and a contrast enhancement state of the first image data.

3. The image processing apparatus according to claim 2, further comprising a subtraction image acquiring unit configured to acquire subtraction image data between the acquired first image data and the acquired comparison image data.

4. The image processing apparatus according to claim 3, wherein the subtraction image acquiring unit is configured to acquire the subtraction image data by calculating the subtraction image data through subtraction operation between the acquired first image data and the acquired comparison image data.

5. The image processing apparatus according to claim 4, wherein the subtraction image acquiring unit is configured to select, based on the comparison image data, at least one of a first subtraction image calculating method or a second subtraction image calculating method which are calculating methods different from each other, and to calculate the subtraction image data through use of the selected at least one of the first subtraction image calculating method or the second subtraction image calculating method.

6. The image processing apparatus according to claim 5, wherein the first subtraction image calculating method and the second subtraction image calculating method include registration processing having characteristics different from each other, and the subtraction image data is calculated based on a result of registration between the first image data and the comparison image data performed by using the registration processing.

7. The image processing apparatus according to claim 2, further comprising a priority setting unit configured to set the priority.

8. The image processing apparatus according to claim 7, wherein the priority setting unit is configured to set the priority of a combination including the first image data and comparison image data in which a date/time difference between a first date/time at which the first examination is performed and a second date/time at which an examination is performed in order to obtain the comparison image data is larger than a predetermined value so as to be higher than the priority of a combination including the first image data and comparison image data in which the date/time difference is smaller than the predetermined value.

9. The image processing apparatus according to claim 7, wherein the priority setting unit is configured to set, in a case of a combination including comparison image data in which a date/time difference between a first date/time at which the first examination is performed and a second date/time at which an examination is performed in order to obtain the comparison image data is larger than a predetermined value, the priority of a combination including comparison image data having a small difference in contrast enhancement state from the first image data so as to be higher than the priority of a combination including comparison image data having a large difference in contrast enhancement state from the first image data.

10. The image processing apparatus according to claim 7, wherein the priority setting unit is configured to set, in a case of a combination including comparison image data in which a date/time difference between a first date/time at which the first examination is performed and a second date/time at which an examination is performed in order to obtain the comparison image data is equal to or smaller than a predetermined value, the priority of a combination including comparison image data having a large difference in contrast enhancement state from the first image data so as to be higher than the priority of a combination including comparison image data having a small difference in contrast enhancement state from the first image data.

11. The image processing apparatus according to claim 7, wherein the priority setting unit is configured to set the priority based on an examination object.

12. The image processing apparatus according to claim 2, further comprising a display control unit configured to perform processing of displaying an image pair including the first image data and the selected comparison image data on a display unit.

13. The image processing apparatus according to claim 3, further comprising a display control unit configured to perform processing of displaying an image pair including the first image data and the selected comparison image data on a display unit,
wherein the display control unit is configured to cause the display unit to display a confirmation screen for confirming whether to acquire the subtraction image data.

14. The image processing apparatus according to claim 12, wherein the display control unit is configured to cause the display unit to display the first image data and the comparison image data so as to be comparable to each other.

15. The image processing apparatus according to claim 3, further comprising a display control unit configured to perform processing of displaying an image pair including the first image data and the selected comparison image data on a display unit,
wherein the display control unit is configured to cause the display unit to display the first image data, the comparison image data, and the subtraction image data so as to be comparable to each other.

16. The image processing apparatus according to claim 12, wherein the display control unit is configured to cause the display unit to display information corresponding to the priority.

17. The image processing apparatus according to claim 1, further comprising a priority acquiring unit configured to acquire priority with respect to a combination including each of candidates for first image data and each of candidates for comparison image data, the comparison image data being image data for performing comparison to the first image data,
wherein the first image acquiring unit configured to acquire the first image data from the candidates for the first image data, in accordance with the priority.

18. An image processing apparatus comprising:
a first image acquiring unit configured to acquire first image data which is image data obtained by imaging a subject by a first examination; and
a comparison image acquiring unit configured to acquire comparison image data for performing comparison to the first image data,
wherein the comparison image acquiring unit is configured to perform at least any one of: 1^{st} acquiring comparison image data including:
attempt to acquire, as the comparison image data, second image data which is image data obtained by imaging the subject by a second examination which is a past examination performed before the first examination; and
acquire, when the second image data has failed to be acquired, as the comparison image data, third image data which is image data different in contrast enhancement state from the first image data, and
2^{nd} acquiring comparison image data including:
attempt to acquire, as the comparison image data, second image data which is image data different in contrast enhancement state from the first image data; and
acquire, when the second image data has failed to be acquired, as the comparison image data, third image data which is image data obtained by imaging the subject by a third examination which is a past examination performed before the first examination.

19. An image processing apparatus comprising:
a first image acquiring unit configured to acquire first image data which is image data obtained by imaging a subject by a first examination;
a comparison image acquiring unit configured to:
acquire, as first comparison image data, image data obtained by imaging the subject under a contrast condition different from a contrast condition of the first image data in the first examination; and
acquire, as second comparison image data, image data obtained by imaging the subject by a second examination performed at an examination date different from an examination date of the first examination;
a subtraction image acquiring unit configured to:
acquire first subtraction image data representing subtraction between the first image data and the first comparison image data; and
acquire second subtraction image data representing subtraction between the first image data and the second comparison image data; and
an evaluating unit configured to perform evaluation of each of the first subtraction image data and the second subtraction image data,
wherein the image processing apparatus is configured to select subtraction image data having a better result of the evaluation from the first subtraction image data and the second subtraction image data.

20. An image processing method comprising the steps of:
acquiring first image data which is image data obtained by imaging a subject by a first examination; and
acquiring comparison image data for performing comparison to the first image data, the comparison image data being acquired in accordance with priority with respect to a combination including the first image data and a candidate for the comparison image data.

21. The image processing method according to claim 20, wherein the priority is configured to be calculated based on an examination date/time of the first examination and a contrast enhancement state of the first image data.

22. The image processing method according to claim 20, further comprising
acquiring configured to acquire priority with respect to a combination including each of candidates for first image data and each of candidates for comparison image data, the comparison image data being image data for performing comparison to the first image data,
wherein the acquiring first image data is performed configured to acquire the first image data from the candidates for the first image data, in accordance with the priority.

23. An image processing method comprising the steps of:
acquiring first image data which is image data obtained by imaging a subject by a first examination; and
acquiring comparison image data for performing comparison to the first image data,
wherein the step of acquiring comparison image data includes at least any one of 1^{st} acquiring comparison image data and 2nd acquiring comparison image data:
1^{st} acquiring comparison image data including:
attempting to acquire, as the comparison image data, second image data which is image data obtained by imaging the subject by a second examination which is a past examination performed before the first examination; and
acquiring, when the second image data has failed to be acquired, as the comparison image data, third image data which is image data different in contrast enhancement state from the first image data, and
2^{nd} acquiring comparison image data including:
attempting to acquire, as the comparison image data, second image data which is image data different in contrast enhancement state from the first image data; and
acquiring, when the second image data has failed to be acquired, as the comparison image data, third image data which is image data obtained by imaging the subject by a third examination which is a past examination performed before the first examination.

24. An image processing method comprising the steps of:
acquiring first image data which is image data obtained by imaging a subject by a first examination;
acquiring, as first comparison image data, image data obtained by imaging the subject in a contrast condition different from a contrast condition of the first image data in the first examination, and acquiring, as second comparison image data, image data obtained by imaging the subject by a second examination performed at an examination date different from an examination date of the first examination;
acquiring first subtraction image data representing subtraction between the first image data and the first comparison image data and second subtraction image data representing subtraction between the first image data and the second comparison image data;
performing evaluation of each of the first subtraction image data and the second subtraction image data; and
selecting subtraction image data having a better result of the evaluation from the first subtraction image data and the second subtraction image data.

25. A non-transitory computer-readable storage medium configured to store a program for causing a computer to execute each of the steps included in the image processing method of claim 20.

26. A non-transitory computer-readable storage medium configured to store a program for causing a computer to execute each of the steps included in the image processing method of claim 23.

27. A non-transitory computer-readable storage medium configured to store a program for causing a computer to execute each of the steps included in the image processing method of claim 24.
